(19) United States
(12) Patent Application Publication
(10) Pub. No.: US 2021/0283098 A1
(43) Pub. Date: Sep. 16, 2021

(54) Methods and Compositions for Treating Pain and Itch

(71) Applicant: YISSUM RESEARCH DEVELOPMENT COMPANY OF THE HEBREW UNIVERSITY OF JERUSALEM LTD., Jerusalem (IL)

(72) Inventor: Alexander Binshtok, Jerusalem (IL)

(21) Appl. No.: 17/258,090

(22) PCT Filed: Jul. 5, 2019
(86) PCT No.: PCT/IB2019/000830

§ 371 Date: Jan. 5, 2021

Related U.S. Application Data
(60) Provisional application No. 62/694,159, filed on Jul. 5, 2018.

(51) Int. Cl. A61K 31/352; A61K 31/445; A61K 31/245; A61K 31/05; A61K 31/165; A61P 23/02

(52) U.S. Cl. CPC A61K 31/352; A61K 31/445; A61P 23/02; A61K 31/05; A61K 31/165; A61K 31/245

(57) ABSTRACT

Provided herein, in one aspect, is a composition for use in the treatment of pain or itch, comprising: an effective amount of at least one blocker of a voltage gated ion channel, said blocker having a pKa of at least 8, and an effective amount of at least one agonist of a TRP channel. Methods and kits are also provided.

### CROSS-REFERENCE TO RELATED APPLICATIONS

This application claims priority to and the benefit of U.S. Provisional Patent Application No. 62/694,159 filed Jul. 5, 2018, incorporated herein by reference in its entirety.

### FIELD

The present disclosure generally relates to methods for treating pain and itch. Also, provided herein are compositions and kits that can be used in such methods.

### BACKGROUND

Pain is the most common symptom for which patients seek medical advice and treatment. Pain can be acute or chronic. While acute pain is usually self-limited, chronic pain persists for three months or longer and can lead to significant changes in a patient's personality, lifestyle, functional ability and overall quality of life (Foley, “Pain,” in Cecil Textbook of Medicine, pp. 100-107 (Bennett and Plum eds., 20th ed. 1996)). Moreover, chronic pain can be classified as either nociceptive or neuropathic. Nociceptive pain includes tissue injury-induced pain and inflammatory pain such as that associated with arthritis.

Neuropathic pain is caused by damage to the peripheral or central nervous system and is maintained by aberrant somatosensory processing. There is a large body of evidence relating activity at vanilloid receptors (Di Marzo et al., “Endovanilloid signaling in pain,” Current Opinion in Neurobiology 12:372-379 (2002)) to pain processing.

Nociceptive pain has been traditionally managed by administering non-opioid analgesics, such as acetylsalicylic acid, choline magnesium trisalicylate, acetaminophen, ibuprofen, fenoprofen, diflusinal, and naproxen; or opioid analgesics, including morphine, hydromorphone, methadone, levorphanol, fentanyl, oxycodone, and oxymorphone. Id. In addition to the above-listed treatments, neuropathic pain, which can be difficult to treat, has also been treated with anti-epileptics (e.g., gabapentin, carbamazepine, valproic acid, topiramate, phenytoin), NMDA antagonists (e.g., ketamine, dextromethorphan), topical lidocaine (for post-herpetic neuralgia), and tricyclic antidepressants (e.g., fluoxetine, sertraline and amitriptyline).

Despite existing pain medicines, there remains, however, a clear need in the art for new methods and compositions useful for treating or preventing pain.

### SUMMARY

In one aspect, provided herein is a composition for use in the treatment of pain or itch, comprising: an effective amount of at least one blocker of a voltage gated ion channel, said blocker having a pKa of at least 8, and an effective amount of at least one agonist of a TRP channel.

A further aspect relates to a method of potentiating a voltage gated ion channel blocker having a pKa of at least 8, the method comprising: providing a blocker of a voltage gated ion channel having a pKa of at least 8, and co-administering with the blocker at least one TRP channel agonist in a subject in need thereof.

In some embodiments, the ion channel can be a sodium or calcium channel. In certain embodiments, the blocker can be selected from one or more of: chloroprocaine, bupivacaine, ropivacaine, tetracaine and procaine.

In some embodiments, the TRP channel can be TRPV1 and/or TRPA1. In some embodiments, the at least one agonist is one or more of: capcaicin, cannabidiol (CBD) and tetrahydrocannabinol (THC), or a derivative of each of the foregoing having substantially the same TRP channel agonist activity.

In one embodiment, the composition can include chloroprocaine and capcaicin. In another embodiment, the composition can include chloroprocaine and cannabidiol. In one embodiment, the composition can include chloroprocaine and tetrahydrocannabinol. In another embodiment, the composition can include chloroprocaine, capcaicin, and cannabidiol.

In some embodiments, the composition is formulated for oral, intravenous, intramuscular, rectal, cutaneous, subcutaneous, topical, transdermal, sublingual, nasal, inhalation, vaginal, intrathecal, epidural, or ocular administration. The composition can be for local or regional anesthesia.

In some embodiments, the agonist and the blocker can be administered simultaneously. In some embodiments, the agonist and blocker are administered sequentially.

Also provided herein is a parametrical composition comprising a pharmaceutically acceptable carrier and any of the composition disclosed herein.

A kit is also provided, comprising, optionally in separate reservoirs, an effective amount of a voltage gated ion channel blocker having a pKa of at least 8 and an effective amount of a TRP channel agonist. The kit can optionally further include instructions for using the blocker and the agonist in a combination anti-pain or anti-itch therapy.

### BRIEF DESCRIPTION OF THE DRAWINGS

FIG. 1. Chloroprocaine does not activate TRPV1 channels.

FIG. 2. Co-application of chloroprocaine with capsaicin does not prolong motor block induced by application of chloroprocaine alone.

FIG. 3. Co-application of chloroprocaine with capsaicin prolongs pain blockade.

FIGS. 4A-4B. Chloroprocaine does not induce toxicity.

FIG. 5. Co-application of chloroprocaine with 1% CBD does not prolong motor block induced by application of chloroprocaine alone.

FIG. 6. Co-application of chloroprocaine with CBD prolongs pain blockade.

### DETAILED DESCRIPTION

Local anesthetics such as lidocaine and articaine act by inhibiting voltage-dependent sodium channels in neurons. These anesthetics block sodium channels and thereby the excitability of all neurons (excitable cells in the cardiovascular system), not just pain-sensing neurons (nociceptors). Thus, while the goal of topical or regional anesthesia is to block transmission of signals in nociceptors to prevent pain, administration of local anesthetics also produces unwanted or deleterious effects such as general numbness from blocking of low threshold pressure and touch receptors, motor deficits from block of motor axons and other complications from blocking of autonomic fibers.

Pain- and itch-related neurons (nociceptors and pruriceptors, respectively) which detect noxious stimuli differ from other types of neurons in expressing (in most cases) the transient receptor potential cation channel subfamily V member 1 (TRPV1) channel, activated by painful heat or by capsaicin, the pungent ingredient in chili pepper. Other relevant types of receptors selectively expressed in various types of pain-related and itch-related neurons are transient receptor potential cation channel subfamily A (ankyrin) member 1 (TRPA1) (both pain and itch) and transient receptor potential cation channel subfamily M (melastatin) member 8 (TRPM8) (cold pain).

EP 331609 discloses a new charged quaternary amine compound used in composition for treating pain, e.g., neuropathic pain, inflammatory pain or nociceptive pain, itch, or neurogenic inflammatory disorder in patient by inhibiting voltage-gated sodium channels. This compound is used to inhibit nociceptor neurons when administered alone or in combination with an agonist of transient receptor potential ion channel (TRP channel-forming receptor).

WO 2011006073 mentions other charged blockers of voltage gated sodium channels being QX-314, N-methyl procaine, QX-222, N-octyl-guanidine, 9-aminoacridine, pancuronium.

In these patent applications the voltage gated ion channel blocker is initially charged, and has no inherent membrane permeability—so it can enter the nociceptor neurons only through the TRP channels—when they open due to activation by their agonist. The entry into the neuron only through the TRP channels provides selective blockage of only those neurons that have TRV receptors, while sparing the undesired blockage of low threshold pressure and touch receptors, motor axons and f autonomic fibers.

Provided herein, in some embodiments, is the surprising discovery that, contrary to the teachings of the above patent applications, it is advantageous not to use charged voltage gated ion channel blockers. In contrast, the conventional charged quaternary nitrogen derivative voltage gated channel blocker (such as QX-314) are neurotoxic due to its action on TRPV1 channels. Charged derivatives of voltage gated channel blockers such as QX-314 produce substantial TRPV1-induced influx of calcium which enters the mitochondria, leading to neuronal cell death. Contrary to conventional wisdom, it is disclosed herein that it is more advantageous to use clinically validated blockers having a high pKa. These blockers have inherent, low membrane permeability through all cell membranes. However, unexpectedly, their selective entry to nociceptive neurons is enhanced when they are administered together with a TRP channel agonist.

Further provided herein is that, in addition to the reduced neurotoxicity, usage of clinically validated blockers provides an additional advantage. Without wishing to be bound by theory, it is believed that treatment with existing clinical blockers with high pKa can produce a two-tier pain reduction system: a first, rapid non-selective low level blockage of all neurons that is caused by the low-level membrane permeable entry of the voltage gated ion channel blocker, and a second, high level and prolonged selective blockage of only nociceptive neurons where higher amounts of the compound enter only those neurons bearing TRP receptors. This two-tier approach is advantageous in clinical application since it will produce short lasting general (including motor) blockade which is necessary during surgery or intervention. After complement of the intervention this general blockade wears off, but prolonged and selective pain blockade remains, preventing postsurgical or inflammatory pain, thus reducing the need for other morphine-based analgesic approaches.

In some embodiments, provided herein is a translational approach for “painless” pain selective anesthesia devoid of neurotoxicity. In one embodiment, this can be achieved by painlessly activating TRPV1 and TRPA1 channels using, e.g., Cannabidiol (CBD) and Tetrahydrocannabinol (THC), well-established potent TRPV1 and TRPA1 agonists as previously demonstrated by De Petrocellis et al (Br J Pharmacol 163, 1479-1494 (2011).

The present disclosure provides, in one aspect, a method, as well as a composition, for reducing pain or itch, the method comprising administering to a subject in need thereof:

(i) an effective amount of at least one blocker of a voltage gated ion channel, said blocker having a pKa of at least 8, and

(ii) an effective amount of at least one agonist of a transient receptor potential (TRP) channel.

In various embodiments, the blocker of (i) and the agonist of (ii) can be two or more different compounds. For example, the composition can include one or more blocker and one or more agonist. In one embodiment, the composition can include one blocker and one agonist. In another embodiment, the composition can include one blocker and two agonists.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs. The following references provide one of skill with a general definition of many of the terms used in this disclosure: Academic Press Dictionary of Science and Technology, Morris (Ed.), Academic Press (1st ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1st ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4th ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Various aspects and embodiments are described in further detail in the following subsections.

Ion Channel Blockers

In some embodiments, the voltage gated ion channel is a sodium or calcium channel.

In some embodiments, the blocker can be a compound that has a pKa of at least 8, preferably at least 9. pKₐ is the negative base-10 logarithm of the acid dissociation constant (Kₐ) of a solution. Thus, in physiological pH (about 7.0) only 2.5% of the compound is uncharged and can permeate through the membrane of all cells blocking all voltage gated ion channels in a very low level. In this case 97.5% of the blocker is charged and therefore membrane impermeant. Activation of TRPV1 channels can produce an option for this charged fraction to enter pain- or itch-related neurons, thus substantially increasing their efficacy and producing differential pain (or itch) selective blockade.

Also, considering this facilitative effect lower dose of blockers could be used to achieve the desired effect.

Examples of such voltage gated ion blockers with pKa of at least 8 include chloroprocaine (pKa=9) which is an ester-type local anesthetic having the following formula:

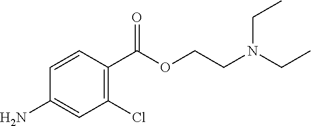 [SMILES: CCN(CC)CCOC(=O)c1ccc(N)cc1Cl]

Other examples of voltage gated ion blockers with pKa of at least 8 include bupivacaine (pKa of 8.1); ropivacaine (pKa of 8.1); tetracaine (pKa of 8.2); and procaine (pKa of 8.9).

TRP Channel Agonists

TRP channel agonists are agonists or activators of transient receptor potential ion channel (TRP channel-forming receptor). The agonist may be an exogenous or endogenous agonist.

Under certain circumstances, TRP channels can be activated in the absence of exogenous TRP channel agonists/ligands by endogenous inflammatory activators that are generated by tissue damage, infection, autoimmunity, atopy, ischemia, hypoxia, cellular stress, immune cell activation, immune mediator production, and oxidative stress. Under such conditions, endogenous molecules (e.g., protons, lipids, and reactive oxygen species) can activate TRP channels expressed on nociceptors, Endogenous inflammatory activators of TRP channels include, for example, prostaglandins, nitric oxide (NO), peroxide (H₂O₂), cysteine-reactive inflammatory mediators like 4-hydroxynonenal, endogenous alkenyl aldehydes, endocannabinoids, and immune mediators (e.g., interleukin 1 (IL-1), nerve growth factor (NGF), and bradykinin, whose receptors are coupled to TRP channels.

The TRP channel may be selected from TRPV1, TRP1A, and P2X. In some embodiments, the TRP channel is TRPV1 or TRP1A.

TRPV1 agonists that can be employed in the methods and kits of the disclosure include capsaicin or other capsaicinoids, which are members of the vanilloid family of molecules. Naturally occurring capsaicinoids include capsaicin itself, dihydrocapsaicin, nordihydrocapsaicin, homodihydrocapsaicin, homocapsaicin, and nonivamide. Other suitable capsaicinoids and capsaicinoid analogs and derivatives for use in the compositions and methods of the present disclosure include naturally occurring and synthetic capsaicin derivatives and analogs including, e.g., vanilloids (e.g., N-vanillyl-alkanedienamides, N-vanillyl-alkanedienyls, and N-vanillyl-cis- monounsaturated alkenamides), capsiate, dihydrocapsiate, nordihydrocapsiate and other capsinoids, capsiconiate, dihydrocapsiconiate and other coniferyl esters, capsiconinoid, resiniferatoxin, tinyatoxin, civamide, N-phenylmethylalkenamide capsaicin derivatives, olvanil, N-[(4-(2-aminoethoxy)-3- methoxyphenyl)methyl]-9Z-octa-decanamide, N-oleyl-homovanillamide, triprenyl phenols (e.g., scutigeral), gingerols, piperines, shogaols, guaiacol, eugenol, zingerone, nuvanil, NE-19550, NE-21610, and NE-28345. Additional capsaicinoids, their structures, and methods of their manufacture are described in U.S. Pat. Nos. 7,446,226 and 7,429,673, which are hereby incorporated by reference.

Additional suitable TRPV1 agonists include but are not limited to eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N- oleoyldopamine), N-arachidonyldopamine (NADA), 6′-iodoresiniferatoxin (6′-IRTX), C18 N- acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxy phenyl]acetamide), JYL79 (N-[2-(3,4-dimethylbenzyl)-3-(pivaloyloxy)propyl]-N′-(4-hydroxy-3-methoxybenzyl)thiourea), hydroxy-alpha-sanshool, 2-aminoethoxydiphenyl borate, 10-shogaol, oleylgingerol, oleylshogaol, and SU200 (N-(4-tert-butylbenzyl)-N′-(4-hydroxy-3-methoxybenzyl)thiourea). Still other TRPV1 agonists include amylocaine, articaine, benzocaine, bupivacaine, carbocaine, carticaine, cyclomethycaine, dibucaine (cinchocaine), dimethocaine (larocaine), etidocaine, hexylcaine, levobupivacaine, lidocaine, mepivacaine, meprylcaine (oracaine), metabutoxycaine, piperocaine, prilocaine, procaine (novacaine), proparacaine, propoxycaine, risocaine, ropivacaine, tetracaine (amethocaine), and trimecaine.

TRP1A agonists that can be employed in the methods, compositions, and kits of the disclosure include any compound that activates TRP1A receptors on nociceptors or pruriceptors and allows for entry of at least one inhibitor of voltage-gated ion channels. Suitable TRP1A agonists include but are not limited to cinnamaldehyde, allyl-isothiocynanate (mustard oil), diallyl disulfide, icilin, cinnamon oil, wintergreen oil, clove oil, acrolein, hydroxy-alpha-sanshool, 2-aminoethoxydiphenyl borate, 4-hydroxynonenal, methyl p-hydroxybenzoate, and 3′-carbamoylbiphenyl-3-yl cyclohexylcarbamate (URB597).

P2X agonists that can be employed in the methods, compositions, and kits of the disclosure include any compound that activates P2X receptors on nociceptors or pruriceptors and allows for entry of at least one inhibitor of voltage-gated ion channels. Suitable P2X agonists include but are not limited to 2- methylthio-ATP, 2′ and 3′-O-(4-benzoylbenzoyl)-ATP, and ATP5′-O-(3-thiotriphosphate).

In some embodiments, the TRP is TRPV1 or TRP1A and the agonist can be cannabidiol (CBD), or a derivative of cannabidiol having the same TRP-agonist activities as CBD, such as cannabidivarin (CBDV), cannabinol (CBN), cannabigerol (CBG), cannabichromene (CBC), and cannabigerolic acid (CBGA). Additional TRPV1 and TRPA2 agonists or activators include tetrahydrocannabinol (THC) or derivatives of tetrahydrocannabinol having the same TRP-agonist activities as THC, such as tetrahydrocannabivarin (THCV).

Cannabidiol is 2-[(1R,6R)-6-isopropenyl-3-methylcyclohex-2-en-1-yl]-5-pentylbenzene-1,3-diol or enantiomers thereof having the following formula:

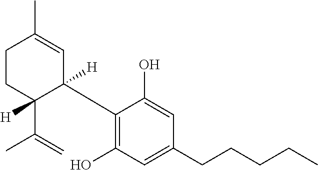 [SMILES: C=C(C)[C@@H]1CCC(C)=C[C@H]1c1c(O)cc(CCCCC)cc1O]

In this connection the term “cannabidiol” applies to a synthetic or an isolated CBD. It should be emphasized that within the context of the present disclosure, this term does not apply to a cannabis, or a crude cannabis or hemp extract, or tinctures of plant material naturally containing CBD. A synthetic CBD can be produced by means of various methods, examples of which methods are described in PCT/IL01/00537 (U.S. Pat. No. 8,071,641), incorporated herein by reference. Pure or a substantially pure CBD can be obtained from a plant material by a number of methods, examples of those are described in U.S. Pat. No. 6,403,123 and Gaoni and Mechoulam (J. Am. Chem. Soc. 93: 217-224 (1971), both incorporated herein by reference.

A “pure” or “isolated” refers to a preparation of CBD with chromatographic purity of 95% or greater, 96% or greater, 97% or greater, 98% or greater, or 99% or greater, or 99.5% or greater, as determined by HPLC, for example. Synthetic or purified CBD can be stored as in a crystalline form or as ethanolic solutions.

From a broader point of view, CBD is a non-psychotropic cannainoid which has a very low affinity for the cannabinoid CB1 and CB2 receptors, but acts as an indirect antagonist of these receptors. Therefore, the terms “synthetic”, “isolated” or “purified” CBD further encompass CBD enantiomers and derivatives displaying the same activity and the same mechanism of action. For example, cannabidiol-dimethylheptyl (also known as CBD-DMH or DMH-CBD) is a synthetic CBD homologue with a replacement of the pentyl chain for a dimethylheptyl chain. This compound is not psychoactive and has similar anticonvulsant and anti-inflammatory to CBD.

Tetrahydrocannabinol (THC) is (−)-(6aR,10aR)-6,6,9-trimethyl-3-pentyl-6a,7,8,10a-tetrahydro-6H-benzo[c]chromen-1-ol and is depicted by the following formula:

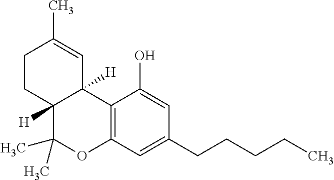 [SMILES: CCCCCc1cc(O)c2c(c1)OC(C)(C)[C@@H]1CCC(C)=C[C@@H]21]

In accordance with the disclosure this term also covers synthetic or isolated THC as well as enantiomers and derivatives displaying the same activity and the same mechanism of action.

Therapeutic Use, Compositions and Kits

In various embodiments, the combination of ion channel blocker(s) and TRP channel agonist(s) can be used to treat or manage pain or itch in a subject in need thereof.

The term “pain” is used herein in the broadest sense and refers to all types of pain, including acute and chronic pain, such as nociceptive pain, e.g., somatic pain and visceral pain; inflammatory pain, postoperative pain, dysfunctional pain, idiopathic pain, neuropathic pain, e.g., centrally generated pain and peripherally generated pain, migraine, and cancer pain. The pains that can be treated by the methods and compositions of the present disclosure include, without limitation, neuropathic pain, inflammatory pain, nociceptive pain, pain due to infections, and procedural pain. The neurogenic inflammatory disorder is selected from the group consisting of allergic inflammation, asthma, chronic cough, conjunctivitis, rhinitis, psoriasis, and inflammatory bowel disease, and interstitial cystitis, atopic dermatitis. The pain may be caused by trauma, surgery, infection and autoimmune diseases. In some embodiments, the pain can be due to a medical procedure such as surgery requiring local or regional anesthesia where nerve block is utilized or due to childbirth, and topical anesthesia for various medicinal and cosmetic procedures.

The term “itch” is used herein in the broadest sense and refers to all types of itching and stinging sensations localized and generalized, acute intermittent and persistent. The itch may be idiopathic, allergic, metabolic, infectious, drug-induced, due to liver, kidney disease, or cancer.

The method may be carried out using all types of administration including: for oral, intravenous, intramuscular, rectal, cutaneous, subcutaneous, topical, transdermal, sublingual, nasal, inhalation, vaginal, intrathecal, epidural, or ocular administration. In some embodiments, the administration is for local or regional anesthesia and is by local injection, epidural, topical, transdermal or ocular.

The subject receiving the administration may be a human subject or an animal for veterinary treatment.

The TRP channel agonist and the voltage gated ion channel blocker (having a pKa of at least 8)-can be administered to the subject simultaneously in one common carrier-composition, may be administered to the subject simultaneously using two separate carrier compositions each suitable for the specific compound, or may be administered sequentially each compound in its own separate carrier.

Thus, in another aspect the present disclosure provides a parametrical composition comprising a pharmaceutically acceptable carrier and a combination of at least one blocker of a voltage gated ion channel, said blocker having a pKa of at least 8, and at least one agonist of TRP channels. The blocker and agonists are as defined above.

In some embodiments, the pharmaceutical composition comprises a pharmaceutically acceptable carrier and (a) a combination of chloroprocaine and cannabidiol or (b) a combination of chloroprocaine and tetrahydrocannabinol.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The composition can be used for the treatment of pain or itch as described above. When administered sequentially the mode of administration may be different—for example while the voltage gated ion channel blocker (having a pKa of at least 8)—such as chloroprocaine, can be administered by local injection, the TRP channel agonist, for example CBD can be administered systemically, for example orally, by inhalation, sub-lingual and the like.

For administration of the two or more compounds each by different carriers (either for sequential or simultaneous administration) the present disclosure further provides a kit comprising two separate dosage forms, each comprising a different agent in its own carrier.

The term “effective amount,” as used herein, refers to that amount of a compound which is sufficient for treating or reducing pain or itch, when administered to a patient. A therapeutically effective amount will vary depending upon the patient and disease condition being treated, the weight and age of the patient, the severity of the disease condition, the manner of administration and the like, which can readily be determined by one of ordinary skill in the art.

Suitable dosage for each compound can range from about 0.01 mg/kg to about 1000 mg/kg (e.g., from about 0.01 to about 100 mg/kg, from about 0.1 to about 50 mg/kg, from about 1 to about 20 mg/kg, from about 1 to about 10 mg/kg) when needed, or regularly (e.g., every 4 to 120 hours), or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970).

In some embodiments, chlorprocaine can be administered at about 0.05% to about 1% (w/v) in concentration, for about 0.1 to about 1000 mL in volume per each administration (e.g., about 0.1 to about 500 mL, about 1 to about 100 mL, about 1 to about 50 mL, about 1 to about 10 mL, about 1 to about 5 mL). In some embodiments, chlorprocaine can be administered at about 0.1 to about 100 mg/kg (e.g., about 1 to about 50 mg/kg, about 1 to about 15 mg/kg, about 1 to about 10 mg/kg, about 5 to about 10 mg/kg, about 10 to about 15 mg/kg). In some embodiments, CBD can be administered at about 0.1 to about 100 mg/kg (e.g., about 1 to about 50 mg/kg, about 1 to about 15 mg/kg, about 1 to about 10 mg/kg, about 1 to about 5 mg/kg, about 5 to about 10 mg/kg). In some embodiments, capsaicin can be administered at about 0.01% to about 0.1% (w/v) in concentration, for about 0.1 to about 1000 mL in volume per each administration (e.g., about 0.1 to about 500 mL, about 1 to about 100 mL, about 1 to about 50 mL, about 1 to about 10 mL, about 1 to about 5 mL).

In yet another aspect, the disclosure provides a kit comprising, in separate reservoirs, an effective amount of the voltage gated ion channel blocker having a pKa of at least 8 (as described above) and an effective amount of TRP channel agonist (as described above), the kit further comprising instructions for using the effective amount of said blocker and effective amount of said agonist in a combination therapy.

The individually or separately formulated agents can be packaged together as a kit. Non-limiting examples include but are not limited to kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

By another aspect the present disclosure provides a method of potentiating the activity of voltage gated ion channel blocker (having a pKa of at least 8), the method comprising co administration of the blocker and at least one TRP channel agonist.

The present disclosure further provides a TRP channel agonist for use in potentiating voltage gated ion channel blocker having a pKa of at least 8.

In some embodiments, the voltage gated ion channel blocker (having a pKa of at least 8) is chloroprocaine and/or the TRP channel agonist is cannabidiol (CBD) or tetrahydrocannabinol (THC).

The term “potentiating” refers to one or more of the following: when administered together with the TRP channel agonist the blocker can be used at lower doses while providing the same effect as compared to the blocker administered alone; and/or when administered together with the TRP channel agonist the blocker can last for longer periods of time as compared to the blocker administered alone; and/or when administered together with the TRP channel agonist the blocker can provide better anti-pain activity as compared to the blocker administered alone.

### EXAMPLES

The following examples, including the experiments conducted and results achieved, are provided for illustrative purposes only and are not to be construed as limiting the disclosure.

### Example 1. The Effect of Chloroprocaine on TRPV1 Channels

To examine the facilitative effect of co-application of TRPV1 activators with blockers with high pKa, we first tested that blockers with high pKa, such as chloroprocaine, does not by themselves, activate TRPV1 channels. To that end, we performed calcium imaging experiments on HEK-293 cells which stably express TRPV1 and TRPA1 using HEK-293t cells for control. The cells were cultured in standard DMEM with 10% FBS, 100 U/ml penicillin and 100 μg/ml streptomycin and 2 mM Glutamax. 5 μg/ml blasticidin and 0.35% Zeocin were added for stable expression of hTRPV1 and for induction tetracycline 0.1 μg/ml was added to the medium 16 to 24 hours prior to experiments. Cells were cultivated at 37° C. and 5% CO₂ and prepared on Poly-D-Lysin coated coverslips and were loaded with Fura-2 AM (stock in DMSO), for 45-60 minutes in a bath solution composed of (in mM): 145 NaCl, 5 KCl, 2 CaCl₂, 1 MgCl₂, 10 Glucose, 10 HEPES. They were then rinsed for 45-60 minutes for de-esterification of intracellular AM esters. Intracellular [Ca²⁺]i was measured fluoroimetrically as ratiometric excitation at 340 nm and 380 nm (ΔF340/380, emission collected at 510 nm) using an inverted microscope (Nikon Eclipse Ti), equipped with an Epi-Fl attachment; perfect focus system (Nikon, Japan) and Exi Aqua monochromator (QImaging). Chroloprocaine (1-5 mM) was bath applied for 120 s. A PV-820 Pneumatic PicoPump (WPI) was used for short (5 s) focal applications of capsaicin. Immediately after the puff application, capsaicin was washed out by perfusing with external solution.

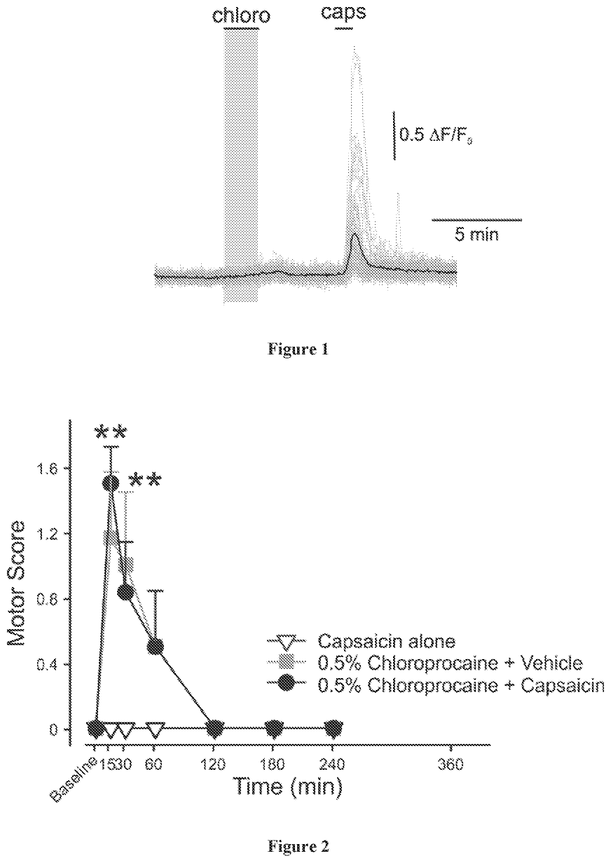

Using this approach, we showed that 120 second application of 1-5 mM of chloroprocaine does not induce calcium influx in hTRPV1-HEK-293 indicating that chloroprocaine does not activate TRPV1 channels (FIG. 1). Specifically, FIG. 2 shows mean (bold) and representative traces (grey) of changes in intracellular calcium in HEK-293 cells stably expressing TRPV1 channels. Note that application of chloroprocaine does not produce any change in FURA-1 fluorescence. Application of capsaicin lead to substantial increase in FURA-2 fluorescence. Representative of 5 out of 5 experiments.

### Example 2. TRP Activators and Chloroprocaine-Induced Selective Pain Blockade

Next we studied if co-application of chloroprocaine and TRPV1 and TRPA1 agonists will lead to differential block. To that end, we performed a series of behavioural experiments first using capsaicin as a TRPV1 activator. To examine the effect of our approach on pain thresholds we injected the drugs separately or their combinations, intraplantary. We used male Sprague-Dawley rats weighing 200-250 grams, which were habituated to handling and behavioral tests prior to the experiments, and tests performed with the experimenter blind to the treatment. Intraplantar injections of vehicle (20% ethanol, 5% Tween 20 in saline; 10 μl), capsaicin (1 μg·μl⁻¹), 2-chloroprocaine (0.5% or 2%) or a mixture of capsaicin and 2-chloroprocaine were made into the left hind paw. The mechanical threshold was determined with an electronic von Frey device (Ugo Basile Biological Research Apparatus) and thermal sensitivity was tested by latency to paw withdrawal from a controlled radiant heat source, Hargreaves test method (Ugo Basile Biological Research Apparatus), focused on an 8×8 mm spot on the plantar surface. Mechanical threshold and thermal paw withdrawal latency were assessed at baseline, and then 15, 30, 60, 120, 180 and 240 minutes post injection.

To study the selectivity of our method, i.e., differential blockade of pain fibers comparing to motor or other sensory fibers, we injected the TRPV1 activator—capsaicin and chloroprocaine peri-sciatically, since sciatic nerve contains both afferent and efferent fibers. For sciatic nerve injections, animals were sedated with medetomidine 100 μg kg⁻¹ and placed in left lateral recumbence. Vehicle, 2-chloroprocaine (0.5% or 2%; 100 μl), alone, capsaicin (50 μg in 100 μl) alone, or 2-chloroprocaine followed by capsaicin (10-min interval) were injected into the area of sciatic nerve below the hip joint on an imaginary line from the greater trochanter to the ischial tuberosity, about one third of the distance caudal to the greater trochanter. Atipamezole at 500 μg·kg⁻¹ was then injected to antagonize sedation. Mechanical threshold was determined with electronic von Frey device, and thermal sensitivity was tested by latency to paw withdrawal from a radiant heat source. Motor function of the injected leg assessed at every time point with a (I) grading score of 0 (no effect; normal gait and limb placement), 1 (limb movement but with abnormal placement and movement) or 2 (complete loss of limb movement) and (II) rotarod test. Moreover, talking, climbing and the placing reflex are examined. Using the “sticky tape” test we examined the effect of this treatment on innocuous sensory sensation as controls in order to show that the blockade is deferential in nature, sparing any sensory or motor functionality. Mechanical threshold, thermal paw withdrawal latency and motor function were assessed at baseline, and then 15, 30, 60, 120, 180 and 240 minutes post injection.

Our first series of experiments which examined whether chloroprocaine can be utilized as a substitution for QX-314 demonstrated that 0.5% chloroprocaine injected alone lead to mild and short-lasting (30 min) motor blockade. Importantly, injection of 0.5% chloroprocaine together with TRPV1 activator, capsaicin, produced motor block which was similar to the motor block induced by the application of 0.5% chloroprocaine alone (FIG. 2). Specifically, FIG. 2 shows mean±SEM of changes in motor scores following peri-sciatic injection of saline, 1 μg/μl capsaicin alone, 0.5% chloroprocaine alone and combination of capsaicin and 0.5% chloroprocaine. Note that application of 0.5% chloroprocaine produced short lasting (30 min) motor block. Importantly, co-application of capsaicin with chloroprocaine does not prolong chloroprocaine-induced block. n=6 rats for each group; two-way ANOVA with post hoc Bonferroni.

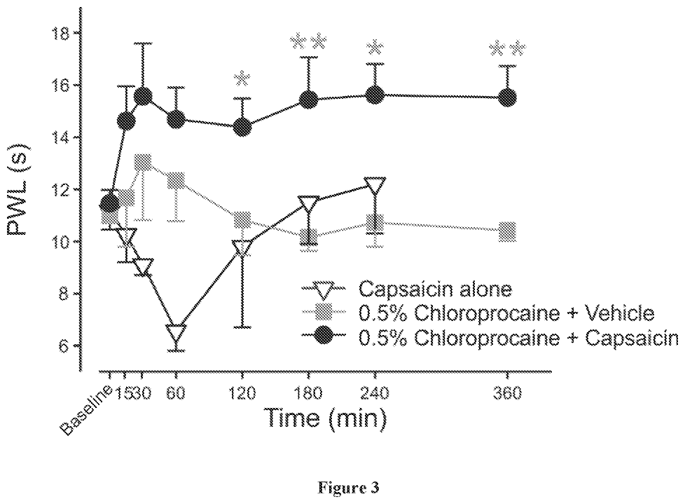

On the other hand, we show that the effect of co-injection of capsaicin with 0.5% chloroprocaine on pain is substantial. Co-injection of 1 μg/μl capsaicin with 0.5% chloroprocaine produced at least 6 hours of pain inhibition (FIG. 3), resulted in 5.5 hours of differential block. Specifically, FIG. 3 shows mean±SEM of changes in latency for paw withdrawal (PWL) from controlled radiant heat source (52° C.), normalized to baseline, after peri-sciatic injections of 1 μg/μl capsaicin alone, 0.5% chloroprocaine alone and combination of capsaicin and 0.5% chloroprocaine. Note that chloroprocaine injected alone does not affect the response to noxious heat. Importantly, co-application of capsaicin with chloroprocaine lead to decreased sensitivity to noxious heat. This partial blockade persists for at least for 6 hours. n=6 rats for each group; two-way ANOVA with post hoc Bonferroni.

The prolonged effect of chloroprocaine when injected with capsaicin suggests that activation of TRPV1 channels provide entry for the charged fraction of chloroprocaine into pain neurons, thus leading to their blockade. The lack of additive effect of chloroprocaine and capsaicin on motor blockade shows that the effect of chloroprocaine is selective for pain neurons.

This data show that chloroprocaine can be used as a substitution of QX-314 and in combination with another TRP channel activator, CBD, could be utilized for pain selective anesthesia.

Moreover, we examined the pain selectivity of our approach in spinal anesthesia. We perform lumbar intrathecal injections under isoflurane anesthesia, the animals' pelvic girdle (whose superior aspect corresponds to the sixth lumbar vertebral body [L6]) is identified by palpation through the skin. The animals are firmly held by the pelvic girdle with one hand. The other hand is used to palpate for the spinous processes of the vertebral column. A 30-gauge, 1-inch disposable needle connected to a microvolume precision syringe (MICROLITER™; Hamilton, Reno, Nev.) is inserted at an angle of approximately 15° relative to the horizontal plane and gently advanced along the groove between the spinous and transverse processes until slipping into the intervertebral space between L5 and L6. The L5-L6 position is selected for injection to minimize the risk of spinal cord injury because of its proximity to the terminal end of the spinal cord in mice older than 120 days. All animals are injected intrathecally with 2 μl of either Vehicle, 2-chloroprocaine (0.5% or 2%) alone, capsaicin (1 μg in 2 μl) alone, or 2-chloroprocaine followed by capsaicin (10-min interval). Mechanical threshold, thermal paw withdrawal latency and motor function are assessed as described for the sciatic nerve block at baseline, and at 30, 60, 120, 240 minutes and 24 hrs post injection.

### Example 3. Examining Chloroprocaine-Induced Neurotoxicity

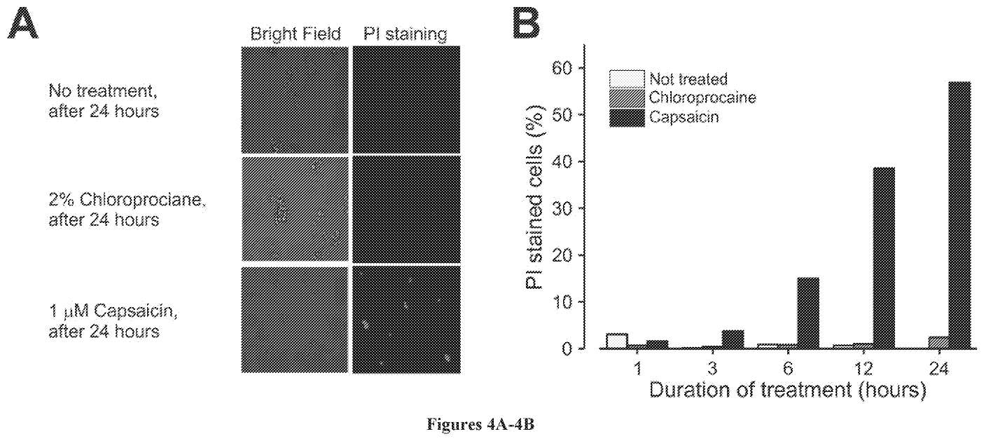

To assure that chloroprocaine does not lead to neurotoxicity we examined cell viability following 1, 3, 6, 12 and 24 hours treatment with 2% chloroprocaine. We assessed cell viability by double staining with PI and fluorescein isothiocyanate (FITC) annexin V. Using this approach, we have demonstrated that 24 hours' treatment with chloroprocaine does not cause cell death (FIGS. 4A-4B). Similar treatment with 1 μg/μl capsaicin lead, after 24 hours, to death of more than 50% of cells. Importantly we previously demonstrated that 24 hour treatment with 1% of charged quaternary amine—QX-314 leads to 55% cell death (see Stueber et al., Anesthesiology, 2016). FIG. 4A shows representative bright field (left) and fluorescent (right) photo-micrographics of TRPV1-HEK-293 cells 24 hours after plating without any treatment (upper panels), 24 hours of 2% chloroprocaine treatment (middle panels) and 24 hours of 1 μM capsaicin treatment (lower panels). Note that treatment with capsaicin lead to extensive PI staining (indicative of cell death), whereas treatment with 2% chloroprocaine did not induce PI staining. Representative of 5 experiments. FIG. 4B is a bar graph summarizing the number of dead TRPV1-HEK-293 cells without any treatment (white), treated with 2% chloroprocaine (grey) or 1 μg/μl capsaicin (black) at the indicated time points. Note that treatment with chloroprocaine does not induce cell death.

In parallel immediately after termination of behavioural experiments in vivo, the animals are anesthetized and perfused via the left ventricle with paraformaldehyde (4% with phosphate-buffered saline) allowing post-mortem immunohistochemical analyses. Sciatic nerves and dorsal root ganglia are removed and placed to fixative. The population of sensory neurons are first labelled using antibodies against TRPV1, IB4 and CGRP, and then counted. The amount of TRPV1, IB4 and CGRP expressing neurons after treatment with TRPA1 activators and QX-314 is compared to the control groups. Neuronal injury is assessed by labelling DRG neurons and axons with antibodies against activating transcription factor-3 (ATF3) and glial fibrillary acidic protein (GFAP). The increase in percentage of ATF-3-immunoreactive cells and hypertrophy or activation of DRG satellite cells (measured by GFAP labelling, which is undetectable in control conditions) have been previously related to lidocaine/QX-314-induced neurotoxicity. Sciatic fibers are section longitudinally and labelled with anti-ATF3 antibodies and antibodies against CD68, a lysosomal protein present in activated macrophages. Then the fibers and cells expressing these labels are counted. Antibodies against neurofilament 200 (NF200 kD) will be used to identify myelinated neurons and antibodies against 510013 will be used to label myelinating Schwan cells. Increased labelling for ATF3 and CD68 compared to control will indicate that the treatment produces neurotoxicity. Recently, long lasting toxic effects of QX-314 together with the TRPV1 agonist, capsaicin, injected in the presence of lidocaine was demonstrated. Therefore, we also examined the long term effect of exposure to capsaicin and chloroprocaine. To that end, animals were injected with the effective concentrations of the drugs twice in intervals of 12 hours. The abovementioned immunohistochemical tests were performed 35 days after the injection.

In Examples 4-6 below, we performed all above-mentioned experiments (effect on pain threshold, differential block, toxicity) but now using CBD or THC, instead of capsaicin to activate TRPV1 and TRPA1 channels.

### Example 4. CBD or THC and Chloroprocaine-Induced Sodium Channel- and Activity Block of Nociceptive Neurons in Culture

We studied the effects of chloroprocaine in combination with CBD/THC on sodium currents and action potential generation in dissociated cultured adult rat DRG neurons. Using calcium imaging, we first identify the cells that express TRPV1 and TRPA1 channels. These cells show increase in intracellular calcium following application of CBD/THC. We then perform whole cell voltage and current clamp recordings from these cells in order to examine the specificity, efficiency and duration of blocking sodium channels and action potentials induced by chloroprocaine alone and chloroprocaine co-applied with (a) CBD (b) THC. We use non-effective doses of chloroprocaine (0.05 mM) and expect that CBP/THC-mediated shuttling of the charged fraction of chloroprocaine into nociceptive neurons will produce substantial inhibition of sodium currents and neuronal excitability. We also use effective dose of chloroprocaine (0.5 mM) and expect that co-application with CBD/THC will prolong the effect of chloroprocaine on sodium currents and neuronal excitability. As a control, we will examine the effect of CBD and chloroprocaine on neurons which do not express functional TRPV1 or TRPA1 channels (identified by Ca′ imaging).

### Example 5. CBD/THC and Chloroprocaine-Induced Acute and Long-Lasting Toxicity

Next, using a repertoire of methods described above we examined if the combination of CBD/THC and chloroprocaine leads to neurotoxicity both in vitro (using PI staining) and in sciatic nerves from animals treated with CBD/THC and chloroprocaine.

### Example 6. CBD/THC and Chloroprocaine-Induced Selective Pain Blockade

We next examined the effect of combination of chloroprocaine with CBD on pain and motor blockade.

To that end, we injected CBD and chloroprocaine peri-sciatically, since sciatic nerve contains both afferent and efferent fibers. For sciatic nerve injections, animals sedated with medetomidine 100 μg kg⁻¹ and placed in left lateral recumbence. Vehicle, 2-chloroprocaine (0.5% or 2%; 100 μl), alone, capsaicin (50 μg in 100 μl) alone, or 2-chloroprocaine followed by capsaicin (10-min interval) were injected into the area of sciatic nerve below the hip joint on an imaginary line from the greater trochanter to the ischial tuberosity, about one third of the distance caudal to the greater trochanter. Atipamezole 500 μg·kg⁻¹ then injected to antagonize sedation. Thermal sensitivity was tested by latency to paw withdrawal from a radiant heat source. Motor function of the injected leg was assessed at every time point with a (I) grading score of 0 (no effect; normal gait and limb placement), 1 (limb movement but with abnormal placement and movement) or 2 (complete loss of limb movement) and (II) rotarod test. Thermal paw withdrawal latency and motor function were assessed at baseline, and then 15, 30, 60, 120, 180, 240 and 360 minutes post injection.

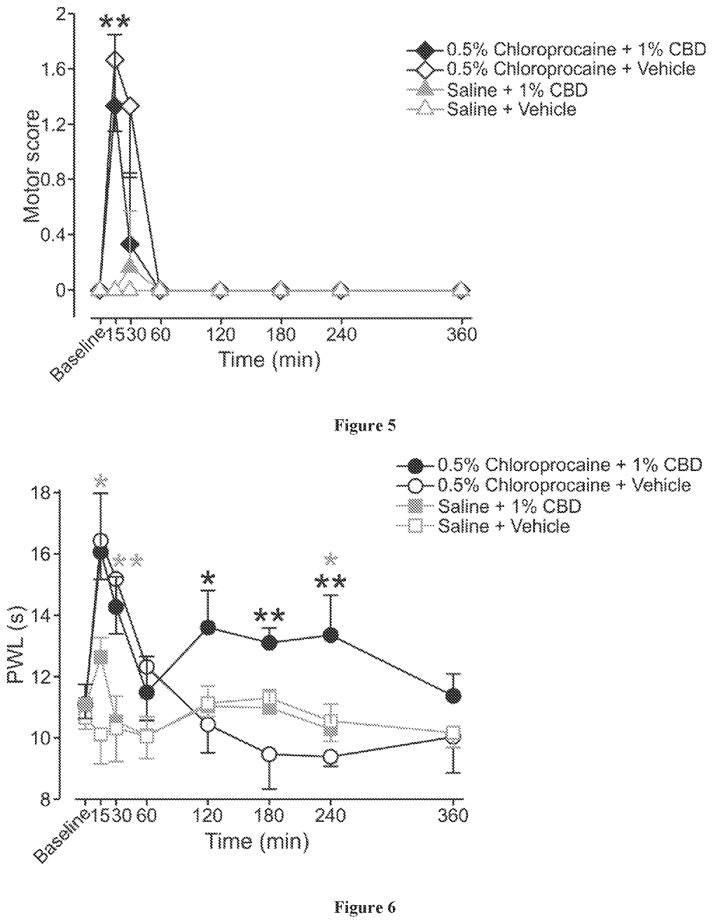

We show that chloroprocaine co-injected with CBD only shortly (15 min) and partially affected motor abilities (FIG. 5). Specifically, FIG. 5 shows mean±SEM of changes in motor scores following peri-sciatic co-injection of (1) saline with vehicle for CBD; (2) saline with 1% CBD; (3) 0.5% chloroprocaine with vehicle for CBD and (4) 0.5% chloroprocaine with 1% CBD. Note that application of 0.5% chloroprocaine produced short lasting (30 min) motor block. Importantly, co-application of CBD with chloroprocaine does not affect chloroprocaine-induced block. n=6 rats for each group; two-way ANOVA with post hoc Bonferroni.

Importantly, co-injection of chloroprocaine and CDB resulted in decrease in responsiveness to thermal painful stimuli which lasted for 4 hours (FIG. 6). Specifically, FIG. 6 shows mean±SEM of changes in latency for paw withdrawal (PWL) from controlled radiant heat source (52° C.), normalized to baseline, after peri-sciatic injections of (1) saline with vehicle for CBD; (2) saline with 1% CBD; (3) 0.5% chloroprocaine with vehicle for CBD and (4) 0.5% chloroprocaine with 1% CBD. Note that chloroprocaine injected with vehicle inhibits the response to noxious heat for about 30 min. Importantly, co-application of CBD with chloroprocaine prolongs the partial blockade of the response to noxious heat till 4 hours. Black asterisk, comparison between “0.5% chloroprocaine with 1% CBD” group to “0.5% chloroprocaine with vehicle for CBD” group. Grey asterisk, comparison between “0.5% chloroprocaine with 1% CBD” group to “saline with vehicle for CBD” group; n=6 rats for each group; two-way ANOVA with post hoc Bonferroni. This effect was significantly longer that the effect of chloroprocaine alone which lasted only 30 minutes. CBD alone at this dose did not affect pain behavior.

Taking together these results show that combination of chloroprocaine with CDB, at these doses, produced almost 4 hours of differential inhibition (i.e., inhibition of response to painful stimuli after the inhibition of motor function has worn off). Similar to our previous results (FIGS. 2 and 3) 0.5% of chloroprocaine alone produced same motor and pain block (no differential block) and CBD did not affect either response to noxious stimuli or motor abilities at all.

In conclusion, our data suggests that co-application of CBD facilitates the effect of chloroprocaine on blocking pain, without affecting motor function. The duration of the differential effect was substantial, and the attenuation of pain was moderate. Further pain reduction can be addressed by experimenting with increased doses of CBD to enhance CBD-mediated TRPV1 opening, thus increasing the possibility of charged fraction of chloroprocaine to enter nociceptive neurons.

### Example 7. Prospective Randomized Clinical Trial on Patients Undergoing Hand Surgery

It is common for patients undergoing hand surgery to receive a high concentration brachial plexus peripheral nerve block to facilitate the surgery and immediate post-operative analgesia. We randomize informed and consented adult patients undergoing hand surgery to 4 groups according to the local anesthetic composition and as to whether CBD was administered: (1) 2% chloroprocaine nerve block with no CBD and (2) 2% chloroprocaine with CBD, We expect a similar onset of action of all blocks and a similar duration of motor block, but expect that where TRPV1 and TRPA1 has been activated (groups 2) analgesia (as pain subjectively reported by the patient and also as measured by pin-prick sensation) will extend longer than where chloroprocaine was given alone.

### Example 8. Protocol for Evaluating the Efficacy of CBD and Chloroprocain for Selective, Long Lasting Local Anesthesia

The aim of this study is to determine the optimal CBD concentration needed to potentiate and elongate the regional anesthetic effect of chloroprocaine in local anesthesia (1. perineural injection (=nerve block) 2. Epidural injection).

### Methods:

Plantar (hind paw) Hargreaves assay as described above.
Motor score assay as described above.
Injection protocol:
   A: Two separate injections. Chloroprocaine will be injected at t=0, CBD will be injected 10 min later.
   B: single injection of chloroprocaine and CBD combined formulation.
      Study groups:
Vehicle
0.05-0.5% chloroprocaine
100 μl from 1-10 mg/ml CBD solution
Combination of chloroprocaine and CBD, in doses within the above ranges.

### Example 9. Protocol for Evaluating the Efficacy of CBD+Chloroprocain+Capcaicin for Selective, Long Lasting Local Anesthesia

The aim of this study is to determine the optimal CBD concentration needed to potentiate and elongate the regional anesthetic effect of chloroprocaine in local anesthesia (1. perineural injection (=nerve block) 2. Epidural injection).

### Methods:

Plantar (hind paw) Hargreaves assay as described above.
Motor score assay as described above.
Injection protocol:
   A: Two separate injections. Chloroprocaine will be injected at t=0, CBD+capcaicin will be injected 10 min later.
   B: single injection of chloroprocaine, CBD and capcaicin combined formulation.
      Study groups:
Vehicle
0.05-0.5% chloroprocaine
100 μl from 1-10 mg/ml CBD solution
0.1-0.5 μM capcaicin
Triple combination of chloroprocaine, CBD and capcaicin in doses within the above range.

### OTHER EMBODIMENTS

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and patent applications referenced in this specification are incorporated herein by reference in their entirety for all purposes to the same extent as if each individual publication, patent or patent application were specifically indicated to be so incorporated by reference.

1. A composition for use in the treatment of pain or itch, comprising:
an effective amount of at least one blocker of a voltage gated ion channel, said blocker having a pKa of at least 8, and
an effective amount of at least one agonist of a TRP channel.

2. The composition of claim 1 wherein the ion channel is sodium or calcium channel.

3. The composition of claim 1 wherein the blocker is selected from the group consisting of: chloroprocaine, bupivacaine, ropivacaine, tetracaine and procaine.

4. The composition of claim 1 wherein the TRP channel is TRPV1 or TRPA1.

5. The composition of claim 4 wherein the at least one agonist is one or more of capcaicin, cannabidiol (CBD) and tetrahydrocannabinol (THC), or a derivative of each of the foregoing having substantially the same TRP channel agonist activity.

6. The composition of claim 1, comprising chloroprocaine and capcaicin.

7. The composition of claim 1, comprising chloroprocaine and cannabidiol.

8. The composition of claim 1, comprising chloroprocaine and tetrahydrocannabinol.

9. The composition of claim 1, comprising chloroprocaine, capcaicin, and cannabidiol.

10. The composition of claim 1, formulated for oral, intravenous, intramuscular, rectal, cutaneous, subcutaneous, topical, transdermal, sublingual, nasal, inhalation, vaginal, intrathecal, epidural, or ocular administration.

11. The composition of claim 10 wherein the composition is for local or regional anesthesia.

12. The composition of claim 1 wherein the agonist and the blocker are administered simultaneously.

13. The composition of claim 1 wherein the agonist and blocker are administered sequentially.

14. A parametrical composition comprising a pharmaceutically acceptable carrier and the composition of claim 1.

15. A kit comprising, in separate reservoirs, an effective amount of a voltage gated ion channel blocker having a pKa of at least 8 and an effective amount of a TRP channel agonist, the kit optionally further comprising instructions for using the blocker and the agonist in a combination anti-pain or anti-itch therapy.

16. A method of potentiating a voltage gated ion channel blocker having a pKa of at least 8, the method comprising: providing a blocker of a voltage gated ion channel having a pKa of at least 8, and co-administering with the blocker at least one TRP channel agonist in a subject in need thereof.

17. The method of claim 16 wherein the blocker is chloroprocaine.

18. The method of claim 16 wherein the at least one TRP channel agonist is one or more of capcaicin, cannabidiol and tetrahydrocannabinol.

19. The method of claim 16 wherein the co-administering comprises administering the agonist and the blocker simultaneously.

20. The method of claim 16 wherein the co-administering comprises administering sequentially.
